(19) United States
(12) Patent Application Publication
(10) Pub. No.: US 2009/0028839 A1
(43) Pub. Date: Jan. 29, 2009

(54) Compositions and methods for the prophylaxis and treatment of vaginal infections

(76) Inventors: Mikhail L. Tchikindas, Princeton, NJ (US); Alla A Aroutcheva, Wheeling, IL (US); Sebastian Faro, Houston, TX (US)

Correspondence Address: Christopher G Izzo;Rutgers University - OCLTT, 3 Rutgers Plaza, ASBILL, New Brunswick, NJ, 08901, US

(21) Appl. No.: 11/794,274

(22) PCT Filed: Dec. 23, 2005
(86) PCT No.: PCT/US2005/047074

§ 371 Date: Sep. 10, 2008

Related U.S. Application Data
(60) Provisional application No. 60/639,192, filed on Dec. 23, 2004.

(51) Int. Cl. A61K 35/74; C12N 1/20; C07K 2/00; A61K 38/02; A61P 31/12

(52) U.S. Cl. 424/93.45; 435/252.9; 530/300; 514/2

(57) ABSTRACT

Applicants have discovered that a particular bacteriocin from the vaginal strain of Lactobacillus rhamnosus, lactocin (160), possesses a particularly advantageous activity against microorganisms associated with vaginal infections, and more specifically those associated with Bacterial Vaginosis (BV). Specifically, this compound is an effective and non-cytotoxic antibacterial agent that provides the basis for formulations MW for the improved prophylaxis and treatment of vaginal infections, and attendant reduction in the acquisition and proliferation of HIV. Applicants have isolated and purified the antimicrobial peptide lactocin (160) from a vaginal strain of L. rhamnosus. The peptide shows antibacterial activity in an agar-well bioassay against the major BV-associated bacteria, G. vaginalis and anaerobes (P bivia and Peptostreptococcus spp.) and does not inhibit the growth of lactobacilli species which are endogenous to a healthy vaginal environment.

### CROSS REFERENCE TO RELATED APPLICATIONS

The present application claims priority under 35 USC § 119(e) to U.S. Provisional Application Ser. No. 60/639192, filed on Dec. 23, 2004, which is incorporated by reference in its entirety herein.

### FIELD OF THE INVENTION

The present invention is directed toward compositions and methods for the prophylaxis and treatment of Bacterial vaginosis and thereby for the reduction in uptake and proliferation of HIV. More particularly, the compositions of the present invention comprise at least one bacteriocin from one or more species of lactobacillus, and even more particularly from Lactobacillus rhamnosus.

### BACKGROUND

Bacterial vaginosis (BV) is a vaginal multi-microbial infection, which increases the risk factor for transmission and acquisition of the human immunodeficiency virus (HIV). An increased rate of HIV infection has been associated with bacterial vaginosis (BV), which is the most common bacterial infection of the vagina in women of reproductive age. The condition is characterized by the replacement of lactobacilli flora with Gardnerella vaginalis and anaerobic bacteria. Although BV is not life threatening, it leads to many adverse effects including preterm birth. It also increases a woman's susceptibility to HIV infection upon exposure to a sexual partner who is infected with the virus. Conventional antibiotic therapy of BV expands the prevalence of drug resistant bacterial strains, and is only effective in 60% of all cases, contributing to a recurrence rate for BV of 30-40%.¹⁻⁴

Several cross-sectional studies identify BV as a cofactor in the sexual transmission of HIV.⁵⁻⁷ The rate of HIV infection among women with BV has been shown to be 2-fold higher than among women with normal vaginal flora.⁸ Females with a higher incidence of BV also have the highest risk of HIV acquisition (50-85%).⁹⁻¹² Applicants' previous in vitro research indicates that the presence of BV-associated bacteria (G. vaginalis and anaerobes) in HIV-infected women is associated with increased HIV RNA levels in the genital tract.^{13,14}

Major BV organisms directly up-regulate HIV replication.^{13, 15-18} G. vaginalis has been isolated in up to 98% of BV cases ¹⁹⁻²⁴, cases and has been found at high concentrations in 60.0% of HIV positive women²⁵. G. vaginalis has been found to increase production of HIV by HIV-infected monocytoid cells and in certain T cells as much as 77-fold.²⁶ Applicants have found that 50% of G. vaginalis strains were able to induce HIV expression in chronically infected U1 cells.¹⁴ Anaerobes, such as P. bivia and P. assaccharaliticus, have also been isolated with high frequency from the vaginas of BV patients ^{21,22,27},and demonstrated high potency in the induction of HIV expression in vitro.¹³ A study in Zimbabwe showed a strong correlation between the absence of vaginal lactobacilli and HIV seropositivity. Studies in Malawi showed that the prevalence of HIV increased from 12% in pregnant women with normal vaginal microflora to 30% among those with BV (both studies were presented at the XI International Conference on AIDS, Vancouver, Canada, 1996). Thus, BV is an aggravating factor leading to the enhanced acquisition of HIV, and accordingly the treatment of BV will ameliorate this exacerbated risk of HIV infection.

The conventional treatment for BV currently recommended by the Centers for Disease Control and Prevention, which utilizes topical administration of the antibiotics metronidazole and clindamycin^{3, 28}, does not eradicate all of the BV-associated bacteria²⁹. After treatment for BV, many women were found to remain colonized by G. vaginalis and/or various BV-associated anaerobes.^{4, 30, 31} Also, the resistance of G. vaginalis to metronidazole was found to increase over one year.³² The high rates of recurrent infection make conventional BV treatment discouraging. Moreover, applicants' in vitro study showed that total inhibition of lactobacilli growth can result with doses of metronidazole and clindamycin that are even lower than topically administered.^{33, 34} Thus, conventional antibiotic-based BV treatments negatively impact the growth of beneficial Lactobacillus species characteristically present in the healthy vaginal environment. This may result in the elimination of healthy vaginal microflora. Therefore, there is a critical need for an antimicrobial agent that will inhibit BV pathogens without killing healthy lactobacilli. Applicants have discovered an alternative therapy based on natural compounds from lactobacilli, which protect the vagina against exogenous and endogenous infection. These compounds are useful in enhancing the efficiency of BV therapy and thus preventing HIV infection, yet do not adversely affect beneficial vaginal microflora.

Lactobacilli are predominant among the microflora in the lower genital tract of healthy females and protect the vagina from invasion by pathogens.^{35, 36} These organisms produce antimicrobial substances, including organic acids, hydrogen peroxide, and bacteriocins, all of which create a hostile environment for pathogens. Applicants' previous in vitro study showed that 59% of the Lactobacillus isolates exhibited all three mechanisms of bacterial antagonism.³⁷ Lactic acid and low pH work synergistically with bacteriocins and are likely more important than hydrogen peroxide for inhibiting the growth of G. vaginalis.³⁸ The acidic environment of the vagina, created primarily by lactobacilli rather than vaginal epithelial cells, has been shown to inhibit HIV.^{17, 39, 40} Replacement of Lactobacillus spp. with BV-associated microorganisms results in an elevation of pH that makes the vaginal environment more favorable for the proliferation of HIV.⁴¹ According to the report of the XI International Conference on AIDS (Vancouver, Canada, 1996), prostitutes in Kenya who had been vaginally colonized with Lactobacillus spp. experimentally had a trend towards a decreased incidence of both gonorrhea and HIV.

Bacteriocins are ribosomally-produced proteinaceous substances of bacterial origin that exhibit antimicrobial activity, and typically consist of 15-50 amino acids.⁴²⁻⁴⁶ Bacteriocins are positively charged molecules with hydrophobic patches that kill sensitive cells by depleting the transmembrane potential (ΔΨ) and/or the pH gradient via the formation of pores in the cell membrane and resultant leakage of cellular materials.⁴⁷ Electrostatic interactions with negatively charged phosphate groups on target cell membranes contribute to the initial binding of many bacteriocins to the cell membrane of sensitive cells.^{48, 49} Other factors, such as the phospholipid composition of the target bacterial membrane, environmental pH, etc., influence a bacteriocin's activity.^{48, 50} For some bacteriocins, “docking molecules” on the target cell membrane facilitate interaction with the antimicrobial peptide, thereby increasing its effectiveness.⁵¹ Bacteriocins exhibit inhibitory effects against various pathogens in a manner similar to antibiotics, however, bacteriocins are distinguishable from antibiotics in terms of their synthesis, mode of action, toxicity and resistance mechanisms.⁵² Organisms resistant to antibiotics are generally not cross-resistant with bacteriocins, and bacteriocin resistance is not always genetically determined.⁵³

Numerous bacteriocins isolated from lactic acid bacteria (LAB) inhibit the growth of food pathogens.^{43, 54-60} The mechanism of action of the bacteriocins nisin and pediocin PA-1 is well studied.^{47, 61} These molecules are active against Gram-positive organisms such as Listeria monocytogenes.^{62, 63} Nisin, a bacteriocin produced by Lactococcus lactis, has an FDA approved GRAS (generally recognized as safe) status for certain applications, and is successfully used for the preservation of various food products in over fifty countries worldwide.⁵³ However, there is currently no LAB bacteriocin-containing product on the market for the prevention and treatment of infections in humans.

Vaginal lactobacilli produce bacteriocins effective against several vaginal pathogens. The peptide extracted from a vaginal isolate of Lactobacillus salivarius inhibited the growth of Enterococcus spp, and Neisseria gonorrhoeae.^{64, 65} Pentocin, isolated from L. pentosus, had a fungistatic effect on Candida albicans.⁴⁶ Applicants previously studied 22 Lactobacillus strains and found that 77.3% exhibited bacteriocin activity against G. vaginalis.³⁷ McLean and McGroaty³² showed that G. vaginalis is highly sensitive to lactobacilli. Although antimicrobially active lactobacilli have been used to develop products for the prevention and treatment of genital infections⁶⁶⁻⁶⁹, the most consumer-accepted product is based on an application of lactobacilli for the production of H₂O₂.^{70, 71} Low vaginal pH is essential for the prevention of vaginal infections, including AIDS. Intravaginal products such as Acidgel, BufferGel, etc. are based on the acid-producing ability of lactobacilli, which helps maintain vaginal pH lower than 4.5.⁷²⁻⁷⁵ However, low vaginal pH alone is insufficient to inhibit vaginal pathogens and prevent infection. Thus far there are no reports on the successful isolation of a lactobacilli strain that can be used for the prevention and treatment of BV.⁷⁶

### BRIEF DESCRIPTION OF THE DRAWINGS

FIG. 1 is a graph showing the levels of HIV p24 (pg/mL) produced by U1 cells stimulated with nine different isolates of Gardnerella vaginalis lysates and a negative control (medium).

FIG. 2 is a photo of an SDS-PAGE gel showing molecular weight markers in lane 1 and the bacteriocin Lactocin 160 in lane 2 with a molecular weight of approximately 3.8 kDa.

FIG. 3 is a graph showing relative luminescence as a measure of extracellular ATP level of Micrococcus luteus ATCC 10240 cells versus incubation time in the presence or absence of the bacteriocin Lactocin 160.

FIG. 4.A. is a graph showing the intracellular ATP concentration in Micrococcus luteus ATCC 10240 cells versus incubation time of such cells in the presence (light circles) or absence (dark circles) of the bacteriocin Lactocin 160.

FIG. 4.B. is a graph showing the extracellular ATP concentration of Micrococcus luteus ATCC 10240 cells versus incubation time of such cells in the presence (light circles) or absence (dark circles) of the bacteriocin Lactocin 160.

FIG. 5.A. is a graph showing the diameter (mm) of the zone of inhibition of growth of Micrococcus luteus ATCC 10240 cells plated in 1.5% nutrient agar (Difco) versus the logarithm of the concentration of the acteriocin Lactocin 160 (mg/ml).

FIG. 5.B. is a graph showing the diameter (mm) of the zone of inhibition of growth of Micrococcus luteus ATCC 10240 cells plated in 1.5% nutrient agar (Difco) versus the logarithm of the concentration of the bacteriocin nisin (mg/ml).

### DESCRIPTION OF THE INVENTION

The present invention embodies applicants' discovery of the unexpectedly advantageous activity of a particular bacteriocin from the vaginal strain of Lactobacillus rhamnosus, lactocin 160, against BV-associated microorganisms. This compound is an effective and non-cytotoxic antibacterial agent that provides the basis for formulations for improvement of both prophylaxis and treatment of BV, and attendant reduction in the acquisition and proliferation of HIV. Applicants have isolated and purified the antimicrobial peptide lactocin 160 from a vaginal strain of L. rhamnosus. The peptide shows antibacterial activity in an agar-well bioassay against the major BV-associated bacteria, G. vaginalis and anaerobes (P. bivia and Peptostreptococcus spp.) and does not inhibit the growth of lactobacilli.

The compositions of the present invention may comprise lactocin 160 either alone or in combination with one or more agents that provide synergistic effects with bacteriocins. These synergistic agents include but are not limited to organic acids, salts of organic acids, saponins, ε-poly-L-lysine, and other agents from lactobacillus. In a preferred embodiment, the composition further comprises lactic acid and/or zinc lactate. In another preferred embodiment, the composition further comprises the saponin Avenacin A-1. Lactocin 160 may be present in its natural form in whole cells of lactobacillus, as a cellular fraction, or in partially or fully purified form, including any pharmaceutically acceptable salts thereof.

The methods of the present invention for the prophylaxis and treatment of BV and HIV include administration of a composition comprising whole cells of lactobacillus or of lactocin 160 in its partially or fully purified form. In a particularly preferred embodiment, a two-stage process is used in which a composition comprising lactocin 160 in its partially or fully purified form is first administered to challenge the BV-associated microflora, followed by the administration of a composition comprising whole cells of one or more of the microflora normally present in a healthy vaginal environment. Thus, the first stage increases the likelihood of strain replacement of BV-associated microorganisms with healthy microflora by easing the burden on the beneficial cells to successfully outcompete the pathogens. Moreover, the second strain replacement stage returns the vaginal environment fully to a healthy state comprising beneficial lactobacillus organisms and accordingly having a low pH antagonistic to further infection with pathogens, including those associated with BV and HIV.

The present invention is described more fully by way of the following non-limiting examples. All references cited in this application, both above and below, are hereby incorporated by reference herein.

### EXAMPLE 1

### Induction of HIV Type 1 Expression by Gardnerella vaginalis and Anaerobes Associated with BV

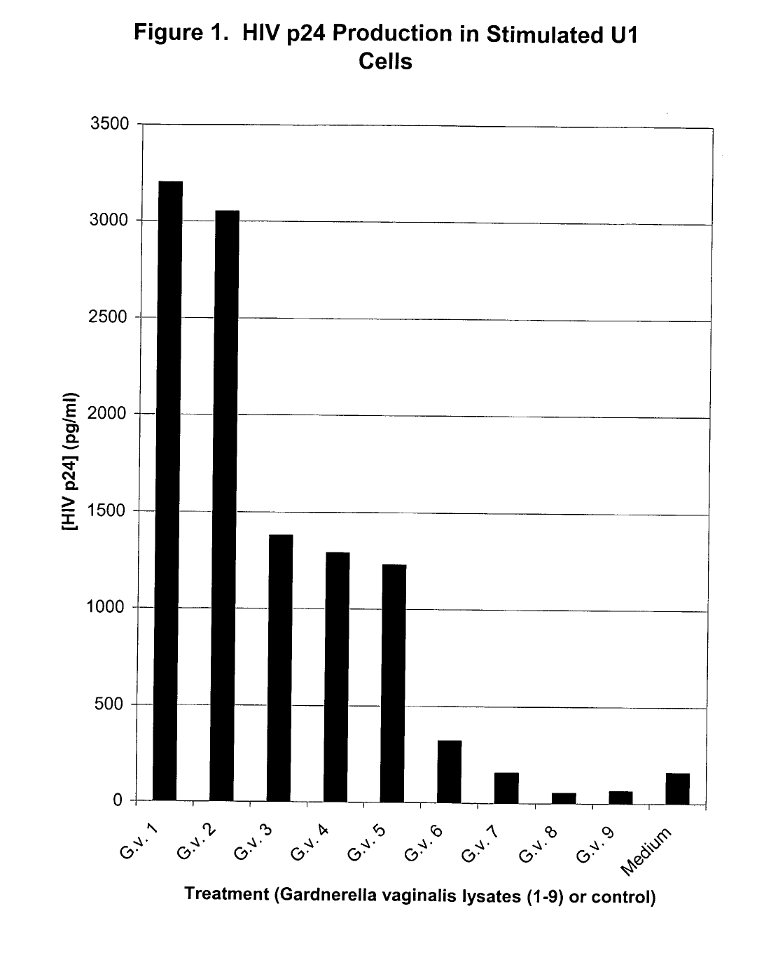

The ability of G. vaginalis to induce HIV expression in vitro was investigated in chronically infected U1 cells by measuring HIV p24 antigen concentration. The addition of lysates from 5 of the 9 G. vaginalis isolates exhibited positive HIV-stimulatory activity (P=0.048), as shown in FIG. 1. Moreover, we have also determined that clinical isolates of G. vaginalis are often resistant to metronidazol (73%), and clindamycin (38%), two drugs of choice for treatment of BV. Finally, we found that 5,000 ng/ml protein from BV-associated P. bivia and P. assaccharolyticus lysate increased HIV expression in eukaryotic cells by 24-fold and 12-fold, respectively. Thus, the major BV-associated pathogens are able to increase sensitivity of human cells to HIV.

### EXAMPLE 2

### Screening for Healthy Vaginal Lactobacilli with Enhanced Antimicrobial Activity

Previously, we studied the ability of 22 Lactobacillus strains to produce antagonistic substances: lactic acid, hydrogen peroxide, and bacteriocins. Lactic acid levels produced by the different strains ranged from 0.68-2.518 mg/ml. More than 81.8% of the strains produced H₂O₂ and 77.3% of the lactobacilli tested produced bacteriocins that inhibited the growth of G. vaginalis.

To test for their antimicrobial activity, all Lactobacillus isolates were grown anaerobically (to eliminate H₂O₂ production) in an MRS broth for 24 hours. The supernatants were collected and filter sterilized. Agar plates (Human Blood Tween agar for G.vaginalis, and Tryptic Soy agar with 5% sheep blood for other organisms) were inoculated with tested bacteria and 400 μl of each supernatant was added into the wells punched in the agar plates. The antimicrobial activity was determined by the size of the inhibition zones, similar to the disk method used for the determination of antibiotic activity. From our collection of clinical lactobacilli strains, we selected the strain identified as L. rhamnosus 160 since its extracellular product was characterized as having the highest antimicrobial activity, as shown in Table 1. We found that this strain produces a bacteriocin that provides exceptional inhibition of the growth of G. vaginalis, an organism that is frequently isolated from BV cases.

TABLE 1
Inhibition of vaginal pathogens by
healthy vaginal Lactobacillus sp.

| Lactobacillus | Group B | Staphylo- | | G. vaginalis |
|---|---|---|---|---|
| (vaginal isolates) | Streptococci | coccus sp. | E. coli | ATCC 14018 |
| strain # | Zone of inhibition (mm) | | | |
| 4 | 4.5 | 5 | 16 | 8 |
| 22 | 4.5 | 7 | 18 | 9 |
| 160 | 12 | 12 | 20 | 12 |
| 161 | 5 | 7 | 17 | 2 |
| 46 | 1 | 4 | 18 | 0 |
| 32 | 8 | 0 | 7 | 1 |
| 135 | 11 | 9 | 20 | 8 |
| 117 | 8 | 6 | 15 | 8 |
| 30 | 9 | 7 | 18 | 5 |
| 114 | 5 | 4 | 20 | 8 |
| 66 | 10 | 8 | 20 | 7 |
| Lactic acid | 1.5 | 1.5 | 1.5 | 1.5 |
| pH 4.0 | | | | |

### EXAMPLE 3

### Isolation of the Antimicrobial Peptide Lactocin 160

The bacteriocin producer L. rhamnosus 160 was isolated from a patient with healthy vaginal microflora. The strain was stored at −70° C. Before use, the cells were cultivated on an MRS agar (Remel, KS) three times under anaerobic conditions at 36° C. The cells were then grown in 2000 ml of MRS broth in an anaerobic chamber for 18 hours at 37° C., after which they were harvested by centrifugation at 7000 × g for 20 min at 5° C. The collected cells were washed three times in 1× phosphate-buffered saline (PBS) (pH 6.0) and transferred into 200 ml of chemically defined media (CDM) (pH 6.0) which mimics normal vaginal secretions with the exception of any protein and amino acid components. The composition of the CDM is shown in Table 2.

TABLE 2
Composition of Chemically Defined Media (CDM) (per 1000 ml).

| Component | Amount | |
|---|---|---|
| NaCl | 3.5 | g |
| KCl | 1.5 | g |
| K₂HPO₄ | 1.74 | g |
| KH₂PO₄ | 1.36 | g |
| Dextrose | 10.8 | g |
| Cystein HCl | 0.5 | g |
| 0.1% Glycogen | 1 | ml |
| 0.03% MgSO₄ | 0.3 | ml |
| 0.004% NaHCO₃ | 0.04 | ml |
| Vitamin K | 1 | ml |
| Nicotinamid | 1 | mg |
| .d-Calcium pantothenate | 1 | mg |
| Biotin | 0.01 | mg |

The cells were incubated at 37° C. for 18 hours under anaerobic conditions with constant shaking and then removed from the media by centrifugation at 12,000 × g for 25 min at 5° C.

Proteins were precipitated from the CDM fraction using 80% ammonium sulfate. The procedure was performed at 5-8° C. by gradual addition of small amounts of ammonium sulfate with continuous, gentle stirring. After 3 hours of incubation at 5° C. , the precipitate was removed by centrifugation at 12,000 × g for 25 min at 5° C. Analysis of the precipitated proteins and the supernatant indicated that the precipitate lacked bioactivity, while the supernatant was found to have antimicrobial activity.

The supernatant was dialyzed against deionized water using a dialysis bag with a molecular weight cut-off (MW CO) of 500 (Spectrum, LA, CA), with four water changes within 3 days. The dialyzed samples were concentrated by lyophilization. The purified preparation of lactocin 160 was completely dissolved in PBS (pH 7.2) to a final concentration of 20 mg/mi.

The sample was subjected to sodium dodecyl sulfate polyacrylamide gel electrophoresis (SDS-PAGE) using 10-20% pre-cast SDS-PAGE gel (Bio-Rad, CA) according to the Bio-Rad manual, using the Tricin buffer at 200V for 30 min. To visualize proteins, the gel was stained with a Bio-Rad Silver Stain, according to the manufacturer's instructions.

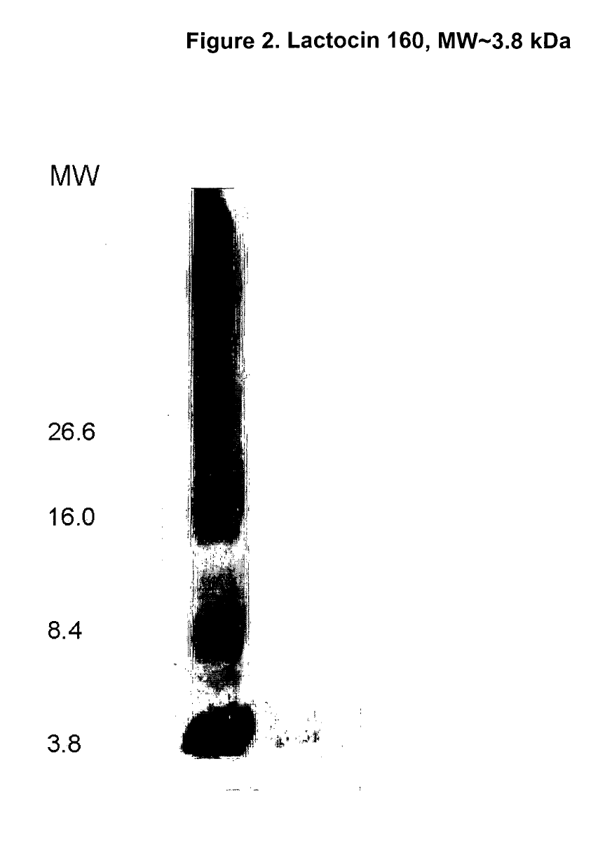

Visualization of the protein bands revealed a separate band with MW of approx. 3.8 kDa, as shown in FIG. 2, which represented the biologically active lactocin 160. The antimicrobial activity of this sample was inhibited by proteinase K, thus confirming the proteinaceous nature of the active agent. According to the SDS-PAGE, lactocin 160 was the only protein present in the sample. Thus, ammonium sulfate precipitation allowed us to separate the bacteriocin from the rest of the proteins excreted by L. rhamnosus 160.

### EXAMPLE 4

### Antimicrobial Activity of Lactocin 160

The sample containing lactocin 160 as obtained from the purification protocol described in Example 3 above was tested against clinical isolates of vaginal pathogens and healthy vaginal Lactobacillus strains. All three Lactobacillus isolates tested were determined to be resistant to the bacteriocin. At the same time, vaginal pathogens were sensitive to lactocin 160 as shown in Table 3. Remarkably, even the most resistant strain of G. vaginalis was sensitive to lactocin 160 at pH 4.5, which is close to the characteristic pH of a healthy vaginal environment (3.8-4.2).

TABLE 3
Susceptibility of vaginal pathogens to lactocin 160

| Microorganism | MIC (μg/ml) | MIC (μg/ml) |
|---|---|---|
| (number of isolates checked) | at pH 6.5 | at pH 4.5 |
| G. vaginalis (3) | 1.5 | <0.2 |
| G. vaginalis (1) | 3 | <0.2 |
| G. vaginalis (3) | 6.2 | <0.2 |
| G. vaginalis (2) | 25 | <0.2 |
| G. vaginalis (1) | 100 | 1.5 |
| Prevotella bivia (1) | 50 | 6.4 |
| Peptostreptococcus sp. (1) | 50 | 6.4 |

(MIC = Minimal Inhibitory Concentration)

### EXAMPLE 5

### Mode of Action of Lactocin 160: ATP Assay

To study the mode of action of lactocin 160, non-vaginal, sensitive cells of Micrococcus luteus ATCC 10240 were treated with lactocin 160 and dissipation of cellular ATP was observed. This particular strain is very sensitive to bacteriocins and thus is commonly accepted as a reference strain for investigations regarding the activity of bacteriocins. An ATP standard curve was constructed by following the procedure described in the manual (Technical Bulletin No. BAAB-1) included with the ATP bioluminescent assay kit (FL-AA, Sigma-Aldrich Corporation; St. Louis, Mo.). The total ATP level of both normal and stressed cells was determined as follows: 1 ml of M. luteus ATCC 10240 was collected by centrifugation, and the pellet was re-suspended in a 50 mM MES (2-(N-Morpholino)-ethanesulfonic acid) buffer (pH 6.5) supplemented with 0.2% glucose and 10 mM KCl to energize the cells.

The cell suspension was divided into 2 aliquots, one to be treated with lactocin 160, and the other to serve as the control (no bacteriocin added). After 10 minutes of energization with glucose, the lactocin 160 preparation was added to the test cell aliquot to yield a final concentration of 10 mg/ml of the preparation. At the same time, an identical volume of the MES buffer was added to the control cells. Test and control samples were then assayed for total and extracellular ATP.

To determine the total ATP level, 20 pi of the cell suspension was quickly added to 80 μl DMSO (dimethyl sulfoxide, Fischer Biotech., Fair Lawn, N.J.) at predetermined time intervals and mixed for 30 seconds before dilution with 4.9 ml of cold water (4° C.). To measure the extracellular ATP level, 20 μl of the same cell suspension was mixed with 80 μl H₂O instead of DMSO. The subsequent steps were the same as the ones for the total ATP determination.

One hundred μl of the mixture of cells with DMSO (for total ATP assay) or H₂O (for extracellular ATP assay) was added to 100 μl of the ATP assay enzyme mix solution (containing luciferase, luciferin, MgSO₄, DTT, EDTA BSA and Tricine buffer salt) (1:25 dilution of stock) and mixed. Relative luminescence intensity (RLI) was determined instantly using a luminometer (Thermo Labsystem, Franklin, Mass.). RLI data were converted into ATP concentration using the ATP standard curve constructed as above. The intracellular ATP level was calculated by subtracting extracellular ATP level from total ATP level.

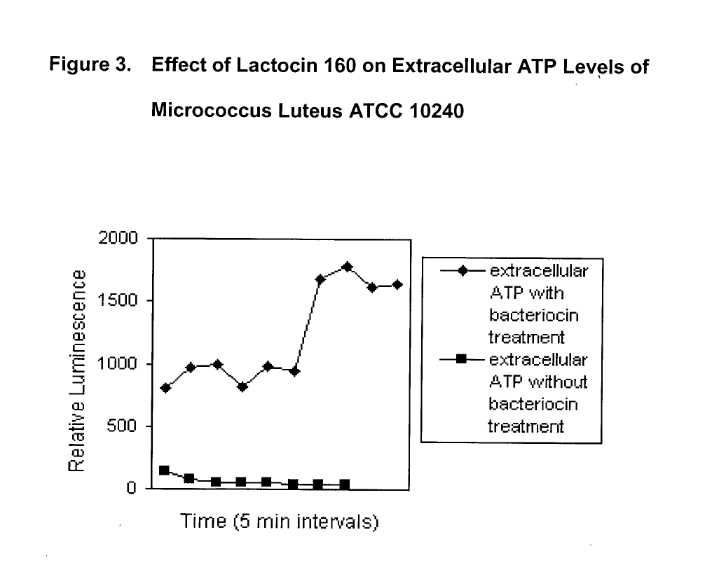

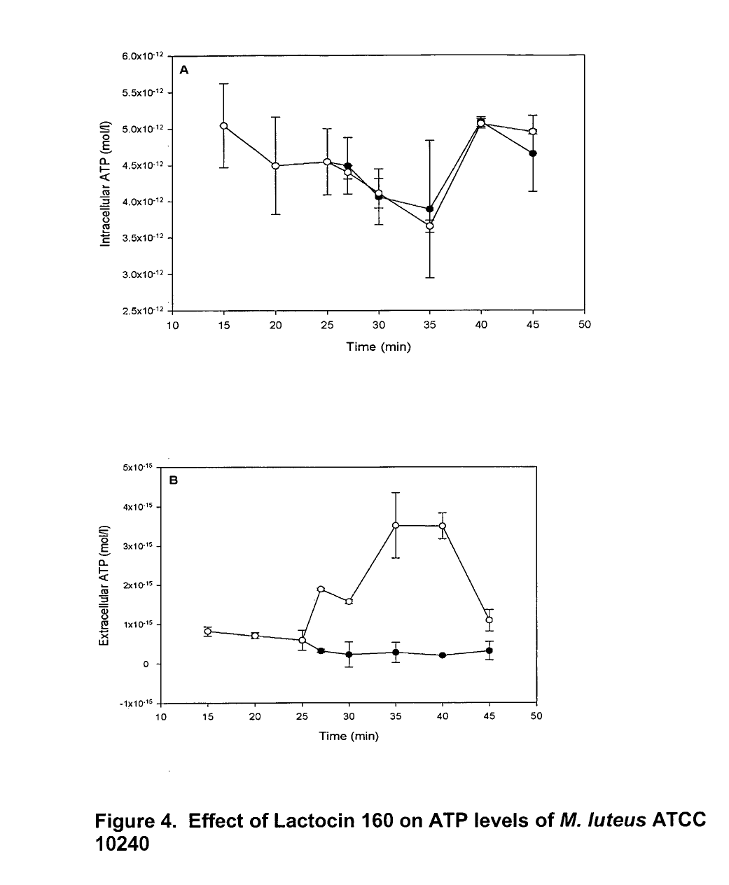

Our results indicate that significant ATP efflux occurs from sensitive cells upon treatment with lactocin 160, as shown in FIG. 3, thus indicating the leakage of cellular components. There were no significant changes in the intracellular ATP level of M. luteus upon the addition of 10 mg/ml of lactocin 160 preparation (final concentration) (FIG. 4A). At the same time, the extracellular ATP level increased significantly (FIG. 4B). This confirms that treatment of the cells with lactocin 160 resulted in an efflux of ATP from within the cells. Lactocin 160 disturbs the cellular membrane and induces ATP efflux. Bacteriocins are known to deplete intracellular ATP, however our results indicate that lactocin 160 induced detectable changes only in the extracellular ATP level.

### EXAMPLE 6

### Mode of Action of Lactocin 160: Membrane Potential (ΔΨ) Dissipation

Nisin powder was obtained from Aplin & Barrett (Beaminster, UK). A high concentration of nisin solution can only be obtained at a low pH since this bacteriocin has optimal solubility in the pH range of 1.7-3.5. To prepare a highly concentrated nisin stock solution, one gram of the dry powder was dissolved in 1 ml of 0.02 N HCl (pH 1.7), a commonly used nisin diluent. The stock solution was used to prepare working concentrations in phosphate-buffered saline (PBS) (pH 7.2). Due to the high buffer capacity of the PBS and the small amounts of nisin in final preparations, the pH of the nisin working solutions was approximately 7.0.

A mid-log phase Micrococcus luteus ATCC 10240 culture was collected by centrifugation and re-suspended in 50 mM HEPES (pH 7.0). The cells were washed twice in the buffer and then re-suspended in a much smaller volume of buffer to achieve OD₆₀₀=40. Fluorescence measurements were performed in a PerkinElmer LS-50B spectrofluorometer (PerkinElmer Life and Analytical Sciences, Inc., Boston, Mass.) using an excitation wavelength of 643 nm, an emission wavelength of 666 nm, and a slit width of 10 nm for both.

For each measurement, 5 μl of a 2 mM solution of the fluorophore DiSC3(5) were added to 2 ml of 50 mM K-HEPES buffer (pH 7.0) to reach a final concentration of 5 μM. After stabilization of the baseline, 20 μl of the M. luteus cell suspension was added (final OD₆₀₀ was ˜0.3), followed by the addition of 20 μl of 20% glucose to energize the cells. Afterward, 2 μl of 5 mM nigericin (to yield a final concentration of 5 μM) was added to convert the pH gradient. After stabilization of the signal, different amounts of nisin (prepared as described above) or lactocin 160 (prepared as described in previous examples) were used to deplete ΔΨ. Finally, 20 μl of 0.3 mM valinomycin (to yield a final concentration of 3 μM) were added to observe complete dissipation of the ΔΨ.

The increase in the fluorescence intensity induced by the different bacteriocins or different concentrations of the same bacteriocin was compared. Fluorescence intensity after the addition of nigericin was denoted as I₀. Intensity upon the addition of either nisin or lactocin 160 was denoted as Iₜ and intensity after the valinomycin-induced total depletion of membrane potential was I∞. Thus, the ΔΨ dissipation % was calculated as:

ΔΨdissipation (%)=(Iₜ-I₀)/ (I∞-I₀)×100

Results are shown in Table 4.

TABLE 4
Comparison of Δψ dissipation in M. luteus
ATCC 10240 by lactocin 160 and nisin

| | |
|---|---|
| Bacteriocin (concentration) | Δψ dissipation % |

lactocin 160

| | | |
|---|---|---|
| 0.4 | mg/ml | 0 |
| 2.0 | mg/ml | 1.83 ± 0.27 |
| 8.0 | mg/ml* | 3.12 ± 0.36 |

nisin

| | | |
|---|---|---|
| 1 | μg/ml | 3.66 ± 0.45 |
| 5 | μg/ml | 18.18 ± 3.10 |
| 10 | μg/ml | 42.35 ± 5.27 |

*8 mg/ml lactocin 160 is equivalent to approximately 13 μg/ml nisin in antimicrobial activity according to the well diffusion assay (see Example 7). In addition, we have found that lactocin 160 has no effect on the membrane ΔpH of M. luteus. Thus, the antimicrobial activity of lactocin 160 cannot be explained solely by dissipation of the membrane potential.

### EXAMPLE 7

### Inhibition of M. luteus ATCC 10240 by Lactocin 160

The antimicrobial activity of lactocin 160 was measured in comparison with that of nisin via a well diffusion assay that monitored the activities of the respective bacteriocins in inhibiting the growth of Micrococcus luteus ATCC 10240 on 1.5% nutrient agar (Difco, MI). The stock culture of M. luteus ATCC 10240 was maintained in a nutrient broth (Difco, MI) with 20% glycerol at −70° C. The strain was streaked on a fresh nutrient agar plate three times (Difco, MI) before it was used as a working culture. The working culture was kept at 4° C. In the assay of lactocin 160, nisin was used as a positive control, while an equal concentration of lyophilized CDM (no bacteriocin; see Example 3, Table 2) was used as a negative control.

Before pouring plates, M. luteus ATCC 10240 cells were seeded into the nutrient agar to a final concentration of approximately 10,000 CFU/ml. When the agar was polymerized in the Petri dishes, small wells of 6 mm in diameter and 4 mm in height were punctured in the agar. Then 80 μl of a 2-fold dilution of the 20 mg/ml preparation of lactocin 160 (see Example 3), the nisin working solution (see Example 6), or the CDM were added into the wells. The agar plates were pre-incubated at 4° C. for 6 hours to allow the bacteriocins to diffuse into the agar, followed by 24 hours incubation at 30° C. The observed inhibition zones indicated activity of the bacteriocins.

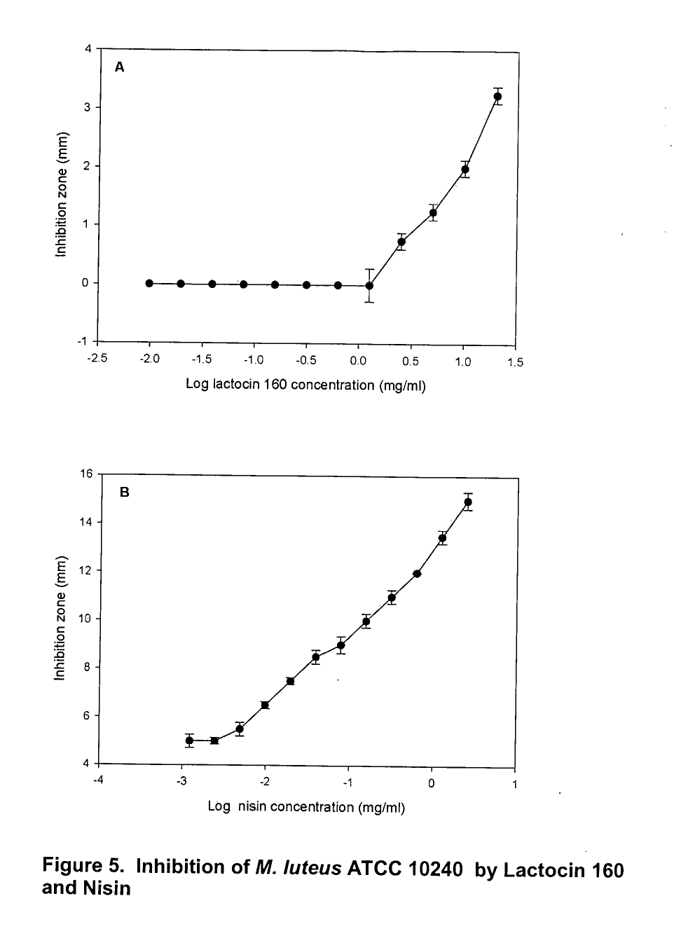

The results are shown in FIG. 5. The minimum inhibitory concentration (MIC) of the lactocin 160 preparation against M. luteus ATCC 10240 was 2.5 mg/ml (FIG. 5A), while that of pure nisin was determined to be 0.25 μg/ml (FIG. 5B). CDM had no effect on the growth of M. luteus ATCC 10240. According to the well diffusion assay, 10 mg of the lactocin 160 preparation was equivalent to the activity of 0.4 μg pure nisin when tested against M. luteus. Most likely, the lower activity of the preparation of lactocin 160 can be explained by the fact that it also contains media components and cellular metabolic products of a non-proteinaceous nature.

References:

1. Bannatyne R M, Smith A M. Recurrent bacterial vaginosis and metronidazole resistance in Gardnerella vaginalis. Sexually Transmitted Infections. 1998;74:455-6.

2. Colli E, Landoni M, Parazzini F. Treatment of male partners and recurrence of bacterial vaginosis: a randomised trial. Genitourinary Medicine 1997;73:267-70.

3. Paavonen J, Mangioni C, Martin M A, Wajszczuk C P. Vaginal clindamycin and oral metronidazole for bacterial vaginosis: a randomized trial. Obstetrics & Gynecology 2000;96:256-60.

4. Boris J, Pahlson C, Larsson P. Six years observation after successful treatment of bacterial vaginosis. Infect Dis Obstet Gynecol 1997;5:297-302.

5. Cohen C R, Duerr A, Pruithithada N, et al. Bacterial vaginosis and HIV seroprevalence among female commercial sex workers in Chiang Mai, Thailand. Aids 1995;9:1093-7.

6. Schmid G, Markowitz L, Joesoef R, Koumans E. Bacterial vaginosis and HIV infection. Sexually Transmitted Infections 2000;76:3-4.

7. Taha T E, Gray R H, Kumwenda N I, et al. HIV infection and disturbances of vaginal flora during pregnancy. Journal of Acquired Immune Deficiency Syndromes 1999;20:52-9.

8. Martin H L, Richardson B A, Nyange P M, et al. Vaginal lactobacilli, microbial flora, and risk of human immunodeficiency virus type 1 and sexually transmitted disease acquisition. Journal of Infectious Diseases 1999; 180:1863-8.

9. Behets F, Andriamiadana J, Rasamilalao D, et al. Sexually transmitted infections and associated socio-demographic and behavioural factors in women seeking primary care suggest Madagascar's vulnerability to rapid HIV spread. Tropical Medicine & International Health 2001 ;6:202-11.

10. Govender L, Hoosen A A, Moodley J, Moodley P, Sturm A W. Bacterial vaginosis and associated infections in pregnancy. International Journal of Gynaecology & Obstetrics 1996;55:23-8.

11. Krohn M A, Hillier S L, Nugent R P, et al. The genital flora of women with intraamniotic infection. Vaginal Infection and Prematurity Study Group. Journal of Infectious Diseases 1995;171:1475-80.

12. Paxton L A, Sewankambo N, Gray R, et al. Asymptomatic non-ulcerative genital tract infections in a rural Ugandan population. Sexually Transmitted Infections 1998;74:421-5.

13. Hashemi F B, Ghassemi M, Faro S, Aroutcheva A, Spear G T. Induction of human immunodeficiency virus type 1 expression by anaerobes associated with bacterial vaginosis. Journal of Infectious Diseases 2000; 181:1574-80.

14. Simoes J A, Hashemi F B, Aroutcheva A A, et al. Human immunodeficiency virus type 1 stimulatory activity by Gardnerella vaginalis: relationship to biotypes and other pathogenic characteristics. Journal of Infectious Diseases 2001; 184:22-7.

15. Al-Harthi L, Roebuck K A, Olinger G G, et al. Bacterial vaginosis-associated microflora isolated from the female genital tract activates HIV-1 expression. Journal of Acquired Immune Deficiency Syndromes 1999;21:194-202.

16. Hillier S L. The vaginal microbial ecosystem and resistance to HIV. AIDS Research & Human Retroviruses. 1998;14 Suppl 1:S17-21.

17. Hill J A, Anderson D J. Human vaginal leukocytes and the effects of vaginal fluid on lymphocyte and macrophage defense functions. American Journal of Obstetrics & Gynecology 1992;166:720-6.

18. Kempf D, Jentsch P, Barre-Sinoussi F B, al e. Inactivation of himan immunodeficiency virus (HIV) by low pH and pepsin. J Acquir Immune Def Syndr 1991;4:828-30.

19. Eschenbach D A. History and review of bacterial vaginosis. American Journal of Obstetrics & Gynecology 1993; 169:441-5.

20. Holst E, Svensson L, Skarin A, Westrom L, Mardh P A. Vaginal colonization with Gardnerella vaginalis and anaerobic curved rods. Scandinavian Journal of Urology & Nephrology. Supplementum 1984;86:147-52.

21. Hillier S, Holmes K. Bacterial vaginosis. In: Holmes Kea, ed. Sexually Transmitted Diseases. New York: McGraw-Hill, 1999.

22. Spiegel C A, Davick P, Totten P A, et al. Gardnerella vaginalis and anaerobic bacteria in the etiology of bacterial (nonspecific) vaginosis. Scandinavian Journal of Infectious Diseases—Supplementum 1983;40:41-6.

23. Totten P A, Amsel R, Hale J, Piot P, Holmes K K. Selective differential human blood bilayer media for isolation of Gardnerella (Haemophilus) vaginalis. Journal of Clinical Microbiology 1982; 15:141-7.

24. Aroutcheva A, Simoes J, Behbakht K, Faro S. Gardnerella vaginalis isolated from patients with BV and those with a normal vaginal ecosystem. Clin Infect Dis 2001;33:1022-7.

25. Mascellino M T, Iona E, Iegri F, et al. Evaluation of vaginal microflora in patients infected with HIV. Microbiologica 1991;14:343-9.

26. Hashemi F B, Ghassemi M, Roebuck K A, Spear G T. Activation of human immunodeficiency virus type 1 expression by Gardnerella vaginalis. Journal of Infectious Diseases 1999; 179:924-30.

27. Hill G B, Livengood C H, 3rd. Bacterial vaginosis-associated microflora and effects of topical intravaginal clindamycin. American Journal of Obstetrics & Gynecology 1994;171:1198-204.

28. Sobel J., Peipert J F., McGregor J A., et al. Efficacy of clindamycin vaginal ovule (3-day treatment) in bacterial vaginosis. Infectious Dis Obtet Gynecol 2001 ;9:9-17.

29. Flores Rivera E, Casanova Roman G, Beltran M, Gonzalez Jimenez M A, Villegas Castrejon H. [Bacterial vaginosis. Relation between the vaginal flora and the vaginal epithelial cells under different treatments. Ultrastructural study]. Ginecologia y Obstetricia de Mexico 1997;65:182-90.

30. Hillier S, Krohn M A, Watts D H, Wolner-Hanssen P, Eschenbach D. Microbiologic efficacy of intravaginal clindamycin cream for the treatment of bacterial vaginosis. Obstetrics & Gynecology 1990;76:407-13.

31. Ferris D G, Litaker M S, Woodward L, Mathis D, Hendrich J. Treatment of bacterial vaginosis: a comparison of oral metronidazole, metronidazole vaginal gel, and clindamycin vaginal cream. Journal of Family Practice 1995;41 :443-9.

32. McLean N W, McGroarty J A. Growth inhibition of metronidazole-susceptible and metronidazole-resistant strains of Gardnerella vaginalis by Lactobacilli in vitro. Applied & Environmental Microbiology 1996;62:1089-92.

33. Aroutcheva A, Simoes J, Shott S, Faro S. The Inhibitory Effect of Clindamycin on Lactobacillus in vitro. Infect Dis Obstet Gynecol 2001 ;9:239-242.

34. Simoes J, Aroutcheva A, Shott S, Faro S. Effect of Metronidazole on the growth of vaginal lactobacilli in vitro. Infect Dis Obstet Gynecol . Infect Dis Obstet Gynecol 2001;9:41-6.

35. Antonio M A, Hawes S E, Hillier S L. The identification of vaginal Lactobacillus species and the demographic and microbiologic characteristics of women colonized by these species. Journal of Infectious Diseases 1999; 180:1950-6.

36. Boris S, Barbes C. Role played by lactobacilli in controlling the population of vaginal pathogens. Microbes & Infection 2000;2:543-6.

37. Aroutcheva A, Gariti D, Simon M, et al. Defense factor of vaginal Lactobacilli. Am J Obstet Gynecol 2001;185(2):375-9.

38. Dembele T, Obdrzalek V, Votava M. Inhibition of bacterial pathogens by lactobacilli. Zentralblatt fur Bakteriologie 1998;288:395-401.

39. Boskey E R, Telsch K M, Whaley K J, Moench T R, Cone R A. Acid production by vaginal flora in vitro is consistent with the rate and extent of vaginal acidification. Infection & Immunity. 1999;67:5170-5.

40. Boskey E R, Cone R A, Whaley K J, Moench T R. Origins of vaginal acidity: high D/L lactate ratio is consistent with bacteria being the primary source. Human Reproduction 2001; 16:1809-13.

41. Taha T E, Hoover D R, Dallabetta G A, et al. Bacterial vaginosis and disturbances of vaginal flora: association with increased acquisition of HIV. Aids 1998;12:1699-706.

42. Carolissen-Mackay V A, G.; Hastings, J. W. Purification of bacteriocins of lactic acid bacteria: problems and pointers. Int.J.Food Microbiol;34:1-16.

43. Contreras B G, De Vuyst L, Devreese B, et al. Isolation, purification, and amino acid sequence of lactobin A, one of the two bacteriocins produced by Lactobacillus amylovorus LMG P-13139. Applied & Environmental Microbiology 1997;63:13-20.

44. Ennahar S, Sashihara T, Sonomoto K, Ishizaki A. Class Ia bacteriocins: biosynthesis, structure and activity. FEMS Microbiology Reviews 2000;24:85-106.

45. Muriana P M, Klaenhammer T R. Purification and partial characterization of lactacin F, a bacteriocin produced by Lactobacillus acidophilus 11088. Applied & Environmental Microbiology 1991;57:114-21.

46. Okkers D J, Dicks L M, Silvester M, Joubert J J, Odendaal H J. Characterization of pentocin TV35b, a bacteriocin-like peptide isolated from Lactobacillus pentosus with a fungistatic effect on Candida albicans. Journal of Applied Microbiology 1999;87:726-34.

47. Chung H J, Montville T J, Chikindas M L. Nisin depletes ATP and proton motive force in mycobacteria. Letters in Applied Microbiology 2000;31:416-20.

48. Chen Y, Ludescher R D, Montville T J. Electrostatic interactions, but not the YGNGV consensus motif, govern the binding of pediocin PA-1 and its fragments to phospholipid vesicles. Applied & Environmental Microbiology 1997;63 :4770-7.

49. Lins L, Ducarme P, Breukink E, Brasseur R. Computational study of nisin interaction with model membrane. Biochimica et Biophysica Acta 1999; 1420:111-20.

50. Chen Y, Shapira R, Eisenstein M, Montville T J. Functional characterization of pediocin PA-1 binding to liposomes in the absence of a protein receptor and its relationship to a predicted tertiary structure. Applied & Environmental Microbiology 1997;63:524-31.

51. Breukink E, Wiedemann I, van Kraaij C, Kuipers O P, Sahl H, de Kruijff B. Use of the cell wall precursor lipid II by a pore-forming peptide antibiotic. Science 1999;286:2361-4.

52. Nissen-Meyer J, Nes I F. Ribosomally synthesized antimicrobial peptides: their function, structure, biogenesis, and mechanism of action. Archives of Microbiology 1997;167:67-77.

53. Cleveland J, Montville T J, Nes I F, Chikindas M L. Bacteriocins: safe, natural antimicrobials for food preservation. Int.J.Food Microbiol 2001;71:1-20.

54. Allison G E, Klaenhammer T R. Functional analysis of the gene encoding immunity to lactacin F, lafI, and its use as a Lactobacillus-specific, food-grade genetic marker. Applied & Environmental Microbiology 1996;62:4450-60.

55. Coventry M J, Muirhead K, Hickey M W. Partial characterisation of pediocin PO2 and comparison with nisin for biopreservation of meat products. International Journal of Food Microbiology 1995;26:133-45.

56. Enan G, el-Essawy A A, Uyttendaele M, Debevere J. Antibacterial activity of Lactobacillus plantarum UG1 isolated from dry sausage: characterization, production and bactericidal action of plantaricin UG1. International Journal of Food Microbiology 1996;30:189-215.

57. Ennahar S, Aoude-Werner D, Sorokine O, et al. Production of pediocin AcH by Lactobacillus plantarum WHE 92 isolated from cheese. Applied & Environmental Microbiology 1996;62:4381-7.

58. Konings W N, Kok J, Kuipers O P, Poolman B. Lactic acid bacteria: the bugs of the new millennium. Current Opinion in Microbiology 2000;3:276-82.

59. Rodriguez J M, Martinez M I, Horn N, Dodd H M. Heterologous production of bacteriocins by lactic acid bacteria. Int.J.Food Microbiol. 2002;80:101-116.

60. Todorov S, Onno B, Sorokine O, Chobert J M, Ivanova I, Dousset X. Detection and characterization of a novel antibacterial substance produced by Lactobacillus plantarum ST 31 isolated from sourdough. International Journal of Food Microbiology 1999;48: 167-77.

61. Montville T J, Chen Y. Mechanistic action of pediocin and nisin: recent progress and unresolved questions. Applied Microbiology & Biotechnology 1998;50:511-9.

62. Chen Y, Montville T. Efflux of ions and ATP depletion induced by pediocin PA-1 are concomitant with cell death in Listeria monocytogenes Scott A. J appl Microbiol 1995;79:684-690.

63. Nilsson L, Chen Y, Chikindas M L, Huss H H, Gram L, Montville T J. Carbon dioxide and nisin act synergistically on Listeria monocytogenes. Applied & Environmental Microbiology 2000;66:769-74.

64. Juarez Tomas M S, Bru E, Wiese B, Ruiz Holgado A A, Nader-Macias M E. Influence of pH, temperature and culture media on the growth and bacteriocin production by vaginal Lactobacillus salivarius CRL 13281. J.Appl Microbiol 2002;93:714-724.

65. Ocana VSPDRH, A. A.; Nader-Macias,M. E. Characterization of a bacteriocin-like substance produced by a vaginal Lactobacillus salivarius strain. Applied & Environmental Microbiology 1999;65:5631-5.

66. Barbes C, Boris S. Potential role of lactobacilli as prophylactic agents against genital pathogens. AIDS Patient Care & Stds. 1999;13:747-51.

67. Famularo G, Pieluigi M, Coccia R, Mastroiacovo P, De Simone C. Microecology, bacterial vaginosis and probiotics: perspectives for bacteriotherapy. Medical Hypotheses 2001;56:421-30.

68. Maggi L, Mastromarino P, Macchia S, et al. Technological and biological evaluation of tablets containing different strains of lactobacilli for vaginal administration. European Journal of Pharmaceutics & Biopharmaceutics 2000;50:389-95.

69. Reid G, Beuerman D, Heinemann C, Bruce A W. Probiotic Lactobacillus dose required to restore and maintain a normal vaginal flora. FEMS Immunology & Medical Microbiology 2001 ;32:37-41.

70. McLean N W, Rosenstein I J. Characterisation and selection of a Lactobacillus species to re-colonise the vagina of women with recurrent bacterial vaginosis. Journal of Medical Microbiology 2000;49:543-52.

71. Ocana V S, Pesce de Ruiz Holgado A A, Nader-Macias M E. Selection of vaginal H2O2-generating Lactobacillus species for probiotic use. Current Microbiology. 1999;38:279-84.

72. Andersch B F, L.; Lincoln,K.; Torstensson, P. Treatment of bacterial vaginosis with an acid cream: a comparison between the effect of lactate-gel and metronidazole. Gynecol.Obstet.Invest 1986;21:16-25.

73. van De Wijgert J, Fullem A, Kelly C, et al. Phase I trial of the topical microbicide BufferGel: safety results from four international sites. Journal of Acquired Immune Deficiency Syndromes & Human Retrovirology 2001;26:21-7.

74. Garg S, Anderson R A, Chany C J, 2nd, et al. Properties of a new acid-buffering bioadhesive vaginal formulation (ACIDFORM). Contraception 2001;64:67-75.

75. Amaral E, Faundes A, Zaneveld L, Waller D, Garg S. Study of the vaginal tolerance to Acidform, an acid-buffering, bioadhesive gel. Contraception. 1999;60:361-6.

76. Reid G. Probiotics for urogenital health. Nutrition in Clinical Care 2002;5:3-8.

We claim:

1. An antimicrobial composition comprising at least one bacteriocin derived from at least one species of Lactobacillus, including pharmaceutically acceptable salts thereof.

2. A method for the prevention and treatment of an infection comprising administration of a therapeutically effective dose of the antimicrobial composition of claim 1.

3. The method of claim 2 wherein the infection is Bacterial Vaginosis.

4. The method of claim 2 wherein the infection is HIV.

5. The antimicrobial composition of claim 1 wherein the at least one species of Lactobacillus comprises Lactobacillus rhamnosus.

6. A method for the prevention and treatment of an infection comprising administration of a therapeutically effective dose of the antimicrobial composition of claim 5.

7. The method of claim 6 wherein the infection is Bacterial Vaginosis.

8. The method of claim 6 wherein the infection is HIV.

9. An antimicrobial composition comprising at least one species of Lactobacillus.

10. A method for the prevention and treatment of an infection comprising administration of a therapeutically effective dose of the antimicrobial composition of claim 9.

11. The method of claim 10 wherein the infection is Bacterial Vaginosis.

12. The method of claim 10 wherein the infection is HIV.

13. The antimicrobial composition of claim 9 wherein the at least one species of Lactobacillus comprises Lactobacillus rhamnosus.

14. A method for the prevention and treatment of an infection comprising administration of a therapeutically effective dose of the antimicrobial composition of claim 13.

15. The method of claim 14 wherein the infection is Bacterial Vaginosis.

16. The method of claim 14 wherein the infection is HIV.
